(19) United States
(12) Patent Application Publication
(10) Pub. No.: US 2005/0240089 A1
(43) Pub. Date: Oct. 27, 2005

(54) Method and computer program for assessing breast cancer risk

(76) Inventor: Colleen Reisz, Prairie Village, KS (US)

Correspondence Address: HOVEY WILLIAMS LLP, Suite 400, 2405 Grand, Kansas City, MO, 64108, US

(21) Appl. No.: 11/111,519

(22) Filed: Apr. 21, 2005

Related U.S. Application Data
(60) Provisional application No. 60/564,128, filed on Apr. 21, 2004.

(51) Int. Cl. A61B 5/00

(52) U.S. Cl. 600/315

(57) ABSTRACT

A method and computer program for assessing breast cancer risk in a patient is disclosed. The method includes determining a cartilaginous volume of at least a portion of an anterior chest wall of the patient, detecting an asymmetry within the cartilaginous volume, and assessing breast cancer risk utilizing the detected asymmetry. The method and computer program are operable to assess breast cancer risk in a non-invasive and time-efficient manner before the actual development or appearance of breast cancer.

### RELATED APPLICATIONS

The present non-provisional patent application claims, with regard to all common subject matter, priority benefit of a co-pending provisional patent application titled NONINVASIVE METHOD AND COMPUTER PROGRAM FOR ASSESSING BREAST CANCER RISK UTILIZING MOLES AND VERTEBRAL ALIGNMENT; application Ser. No. 60/564,128; filed Apr. 21, 2004. The identified provisional patent application is hereby incorporated by reference into the present non-provisional patent application.

### BACKGROUND OF THE INVENTION

1. Field of the Invention

The present invention relates to a method and computer program for assessing breast cancer risk in a patient. More particularly, the invention relates to a method and computer program for assessing breast cancer risk in a patient based upon detecting an asymmetry within at least a portion of a cartilaginous volume of the patient's anterior chest wall.

2. Description of the Related Art

Breast cancer is one of the leading causes of death in women and its incidence is increasing dramatically. As hereditary forms of breast cancer account for only a small minority of women with breast cancer, various conventional methods of detecting and assessing breast cancer risk have been developed in an attempt to detect or predict breast cancer in sufficient time for treatment.

Unfortunately, these conventional methods are generally invasive, time-consuming, or are unable to assess a risk of breast cancer until cancer actually develops. For example, conventional assessing methods include cumbersome sensors and bra-like devices that must be worn by a patient during prescribed times of the patient's menstrual cycle to detect thermal responses of the breast. These cumbersome sensors must be physically adhered to the breast and are uncomfortable, labor intensive, and time sensitive. Such disadvantages inhibit actual use of these devices.

Mammography, MRI, PET are commonly used for detecting breast cancer. These devices are useful for detecting breast cancer only once it has developed and also carry the biologic burden of radiation exposure. Similarly, breast self exams discover breast cancer only when the cancer has grown to a certain size. Thus, known breast-cancer detection methods are targeted only to recognize disease once it has started, thereby foreclosing the application of possible treatments.

### SUMMARY OF THE INVENTION

The present invention solves the above-described problems and provides a distinct advance in the art of assessing breast cancer risk. More particularly, the invention relates to a method and computer program for assessing breast cancer risk in a patient based upon detecting an asymmetry within at least a portion of a cartilaginous volume of the patient's anterior chest wall. The present invention is operable to assess breast cancer risk in a non-invasive and time-efficient manner before the actual development or appearance of breast cancer.

In one embodiment, the invention provides a method for assessing breast cancer risk in a patient. The method includes determining a cartilaginous volume of at least a portion of an anterior chest wall of the patient, detecting an asymmetry within the cartilaginous volume, and assessing breast cancer risk utilizing the detected asymmetry such that a greater asymmetry results in a greater breast cancer risk.

In another embodiment, the method includes determining a cartilaginous volume of at least a portion of a left anterior chest wall of the patient and determining a cartilaginous volume of at least a portion of a right anterior chest wall of the patient. The cartilaginous volumes are compared and breast cancer risk is assessed utilizing the comparison.

In another embodiment, the method includes determining a cartilaginous volume of at least a portion of a left anterior chest wall of the patient and determining a cartilaginous volume of at least a portion of a right anterior chest wall of the patient. A displacement of a sternal prominence and an anteroposterior displacement are also determined. A variance is calculated by comparing the cartilaginous volume of the left anterior chest wall and the cartilaginous volume of the right anterior chest wall and ascertaining the magnitude of the determined displacements such that the calculated variance represents asymmetry within the cartilaginous volumes of the anterior chest wall. Breast cancer risk is assessed utilizing the calculated variance such that a greater variance results in a greater breast cancer risk

In another embodiment, the present invention provides a computer program for assessing breast cancer risk in a patient. The computer program is stored on a computer-readable medium for operating a computing device and includes a code segment operable to determine a cartilaginous volume of at least a portion an anterior chest wall of the patient. The program also includes a code segment operable to detect an asymmetry within the cartilaginous volume and a code segment operable to assess breast cancer risk utilizing the detected asymmetry.

Other features of the present invention will be apparent from the following detailed description of the preferred embodiments and the accompanying drawing figures.

### BRIEF DESCRIPTION OF THE DRAWING FIGURES

A preferred embodiment of the present invention is described in detail below with reference to the attached drawing figures, wherein:

FIG. 1 is a perspective view of equipment that may be utilized by the method and computer program of the present invention shown with an exemplary patient;

FIG. 2 is a schematic view showing various regions of the patient's anterior chest wall;

FIG. 3 is a schematic view showing a cross-section of some of the various regions illustrated in FIG. 2;

FIG. 4 is a schematic view showing an axial view of the patient's anterior chest wall;

FIG. 5 is a schematic view showing a sagittal view of the patient's anterior chest wall; and

FIG. 6 is a flow chart showing some of the steps utilized by the method and computer program of the present invention.

The drawing figures do not limit the present invention to the specific embodiments disclosed and described herein. The drawings are not necessarily to scale, emphasis instead being placed upon clearly illustrating the principles of the invention.

### DETAILED DESCRIPTION OF THE PREFERRED EMBODIMENTS

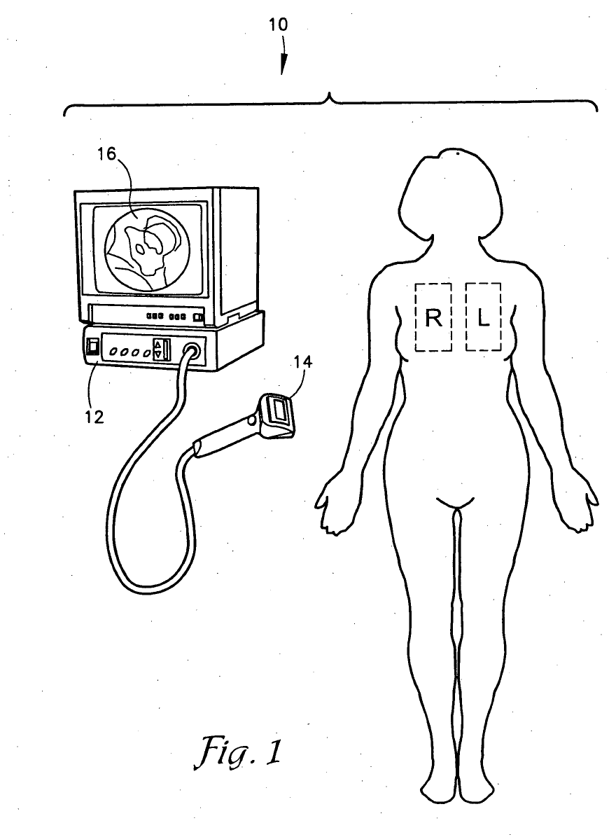

The computer program and method of the present invention for assessing breast cancer risk in a patient may be implemented with the equipment 10 as shown in FIG. 1. The equipment 10 may include computing devices, medical devices, computer software, hardware, firmware, or any combination thereof. In a preferred embodiment, however, the equipment 10 includes a computing device 12 such as a personal computer, a network computer, a computer network comprising a plurality of computers, a mainframe or distributed computing system, a portable computing device, a computing device embedded or internal to medical diagnostic equipment such as radiology equipment, or any combination thereof. The computing device 12 preferably includes computer-readable memory for storing data.

The equipment 10 also includes a detecting device 14 for detecting cartilaginous regions within an anterior chest wall of the patient. Preferably, the detecting device 14 is a ultrasound device. However, the detecting device 14 may be any device or combination of devices that are operable to determine cartilaginous volumes of the anterior chest wall as discussed below. For example, the detecting device 14 may include various radiological, thermal, or electrical elements that are operable to detect cartilaginous regions by differentiating cartilage from bone and/or other elements within the patient's chest in a generally conventional manner.

The detecting device 14 is preferably coupled with the computing device 12 such that data may be exchanged between the devices 12, 14. For example, the detecting device 14 and the computing device 12 may be coupled by cable or wireless link to enable data generated by the detecting device 14 to be stored, analyzed, and processed by the computing device 12 as described below.

The equipment 10 may also include a display 16 for displaying relevant information and stored data. The display 16 is coupled to the computing device 12 in a conventional manner such that the information and data generated by the computing device 12 and/or detecting device 14 may be displayed on the display. For example, the display 16 may provide a real-time indication of the results of the method described below, including cartilaginous volumes, variances, and assessed risks.

Furthermore, the equipment 10 may be integral within one housing, such that the computing device 12, the detecting device 14, and the display 16 are contained within the same housing. Such a configuration may be preferable as it enables the method and computer program of the present invention to be readily portable and utilized on many patients without transporting multiple units or duplicating equipment. However, the computing device 12, detecting device 14, and display 16 may be housed separately and/or at remote locations to utilize pre-existing equipment.

The equipment 10 and computer program illustrated and described herein are merely examples of devices and programs that may be used to implement the present invention and may be replaced with other devices and programs without departing from the scope of the present invention.

The computer program described herein controls operation of the equipment 10. The computer program is stored in or on a computer-readable medium residing on or accessible by the equipment 10, such as within the computing device 12, for instructing the equipment 10 and the other related components to operate as described herein. The computer program preferably comprises an ordered listing of executable instructions for implementing logical functions in the equipment 10.

The computer program can be embodied in any computer-readable medium for use by or in connection with an instruction execution system, apparatus, or device, such as a computer-based system, processor-containing system, or other system that can fetch the instructions from the instruction execution system, apparatus, or device, and execute the instructions.

A “computer-readable medium” can be any means that can contain, store, communicate, propagate or transport the program for use by or in connection with the instruction execution system, apparatus, or device. The computer-readable medium can be, for example, but not limited to, an electronic, magnetic, optical, electromagnetic, infrared, or semi-conductor system, apparatus, device, or propagation medium. More specific, although not inclusive, examples of the computer-readable medium would include the following: an electrical connection having one or more wires, a portable computer diskette, a random access memory (RAM), a read-only memory (ROM), an erasable, programmable, read-only memory (EPROM or Flash memory), an optical fiber, and a compact-disc (CD), a digital video disc (DVD), and similar elements. The computer-readable medium could even be paper or another suitable medium upon which the program is printed, as the program can be electronically captured, via for instance, optical scanning of the paper or other medium, then compiled, interpreted, or otherwise processed in a suitable manner, if necessary, and then stored in a computer memory.

The functionality and operation of a preferred implementation of the computer program is described below. In this regard, some of the described functionality may represent a module segment or portion of code of the computer program of the present invention which comprises one or more executable instructions for implementing the specified logical function or functions. In some alternative implementations, the functions described may occur out of the order described below. For example, functionalities described in succession may in fact be executed substantially concurrently, or the functionalities may sometimes be executed in the reverse order depending upon the functionality involved. Additionally, portions of the computer program and method may be implemented without the use of the computing equipment 10, as described in more detail below.

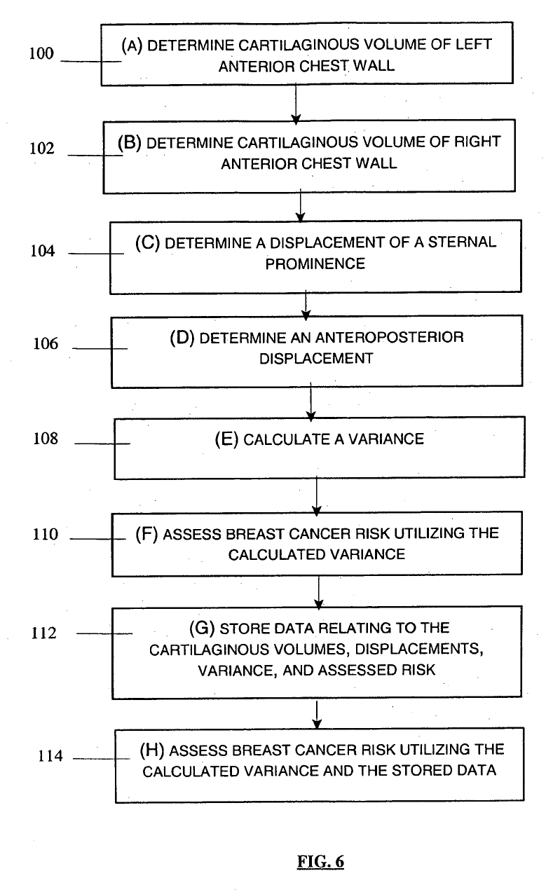

Referring to FIGS. 1-6, the computer software and method of the present invention preferably includes: (a) determining a cartilaginous volume of a left anterior chest wall of the patient, referenced at step 100 in FIG. 6; (b) determining a cartilaginous volume of a right anterior chest wall of the patient, referenced at step 102 in FIG. 6; (c) determining a displacement of a sternal prominence of the patient, referenced at step 104 in FIG. 6; (d) determining an anteroposterior displacement of the patient, referenced at step 106 in FIG. 6; (e) calculating a variance, referenced at step 108 in FIG. 6; (f) assessing breast cancer risk utilizing the calculated variance, referenced at step 110 in FIG. 6; (g) storing data relating to the cartilaginous volumes, displacements, variance, and assessed risk, referenced at step 112 in FIG. 6; and (h) assessing breast cancer risk utilizing the calculated variance and the stored data, referenced at step 114 in FIG. 6.

Patients with cancer risk display risk and exhibit a biomarker at the cartilaginous processes at specific areas in the thorax. Such a biomarker defines those women who have molecular vulnerability in the genes associated with bony alignment in embryogenesis and how that vulnerability can become manifest under the control of the hormonal cascade of puberty. This biomarker may be visible and measurable by the end of a patient's second decade.

There are many classes of genes that have important roles not only during development and embryogenesis but also during life. The Homeobox genes exemplify this process. The Homeobox genes are responsible for anteroposterior alignment, organ fate and cellular integrity of the breast. Each organ system has its own Homeobox molecular portrait. Breast cancer has characteristic Homeobox profiles that are different from normal breast tissue. The alteration of note is focused on the cartilage of endochondral insertions on the center of the anterior chest. The genes responsible for breast cancer are generally related to the genes that set up the bony thorax, specifically the areas of chondrification. The chondrification process is responsible for connecting the sternum with the manubrium and the rib insertions with sternum. These processes mature as women develop their adult body habitus. The hormonal cascade controls the maturation and volume of cartilage. This hormonal cascade ripens and matures the cartilage.

Maturation of cartilage is under hormonal control. For example, gender may be determined from the calcification of the costal cartilages. Other examples of cartilage and hormonal relationships exist, such as the relation of early extensive ossification of costal cartilages to adrenogenital syndrome, the swelling of the second costal cartilage to Tietze's syndrome, and delayed fusion of sternal segments to cretinism and other endocrine disorders. The hormonal influences on cartilage and the resulting asymmetry unmasks genetic differences in homeobox, bone morphogenetic proteins, and other genes responsible for cartilaginous skeletal maturation and formation of the female thoracic phenotype.

Chondrification begins in the seventh week of gestation and completely surrounds the thoracic wall by the end of the fourth month of fetal growth. Rib cartilage is of mesodermal origin, breast is of central ectoderm. PAX-3 and Mtfh-1 (mesenchymal forkhead) are important in the formation of the sternal component of the ribs. The sternum, manubrium, and ribs are initially aligned in the proper position. Asymmetry results from hypertrophy, unilateral distortion, bifidism, and fusion of cartilage.

A partial list of the genes responsible for endochondral differentiation are the BMP, GDF-5, Smads, and Runx2. There are other genes and co-factors that navigate the end result of cartilage formation. BMPs are a large family of secreted growth factors that control events in the early embryonic phase. BMP signaling gradients establish the dorsoventral body axis. BMPs and Hox genes both contribute to multiple processes: early embryonic development; organogenesis; and adult tissue homeostasis and cellular integrity.

Ribs are formed from mesoderm, breast is formed from ectoderm and sternum is formed from the somatopleural mesoderm. Epidermal differentiation is an induced fate, allowed by the secretion of Noggin, Chordin, and Follistatin, all of which block BMP signaling and lead to neural differentiation. Smad activity controls BMP activated proteins and activates epidermal specific gene expression on the anterior chest wall. The set of genes that establish the thoracic phenotype also serve as ligands for hormonal growth during puberty. Distortion in the thoracic phenotype at specific points is predictive for breast cancer.

Thus, the determination of cartilaginous volumes within the anterior chest wall of the patient may be utilized to assess the patient's risk of breast cancer, as the chondrification process and cartilaginous asymmetry between the left anterior chest wall and the right anterior chest wall are molecularly linked to the occurrence of breast cancer. Evaluation of axial variance at the sternal prominence and sagittal assessment of the sternal and xyphoid angles also may contribute to the assessment of risk by determining asymmetry within the anterior chest wall.

In steps 100 and 102, the cartilaginous volumes of the left anterior chest wall, referenced by region L in FIG. 1, and right anterior chest wall, referenced by region R in FIG. 1, are determined. Preferably, the detecting device 14 is utilized to determine the volumes, such as by applying the detecting device 14 to the patient and utilizing the computing device 12 to determine the left and right cartilaginous volumes. The detecting device 14 detects cartilaginous regions in a generally conventional manner by differentiating cartilaginous regions from non-cartilaginous regions, such as by differentiating bone from cartilage. For example, the detecting device 14 may detect cartilaginous volumes in a conventional manner based upon the various densities of the anterior chest wall through ultrasound or other similar methods.

Additionally, the detecting device 14 may itself determine cartilaginous volumes or the detecting device 14 may provide data to other elements or devices, such as the computing device 12, to enable the other elements or devices to determine the cartilaginous volumes. For example, the detecting device 14 may detect various patient physical properties and then provide the detected properties to the computing device 12 to determine cartilaginous volumes.

The determined cartilaginous volumes may be any cartilage region present within the patient's chest. Thus, the cartilaginous volumes of the left anterior chest wall may be any cartilaginous volume present in the left region of the patient's chest while the cartilaginous volumes of the right anterior chest wall may be any cartilaginous volume present in the right region of the patient's chest.

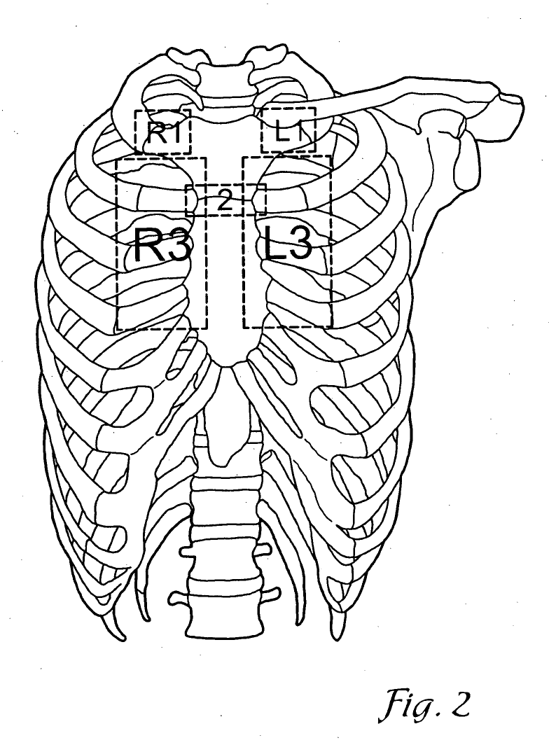

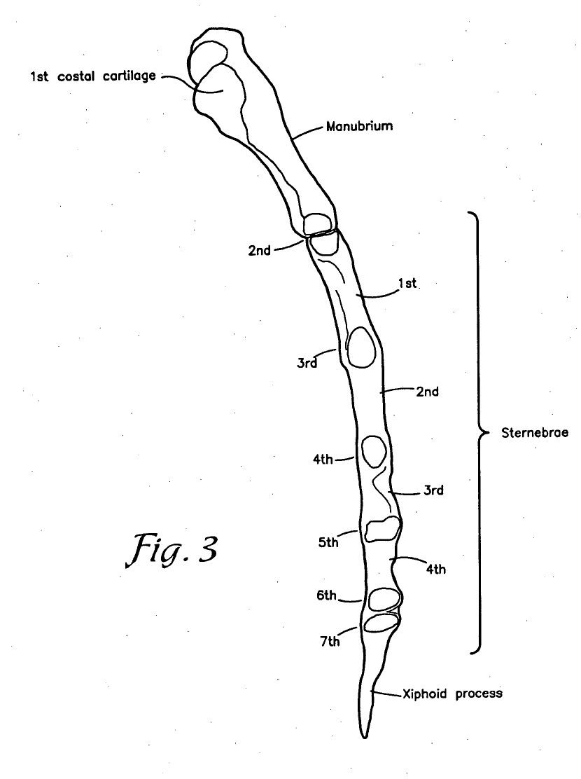

As shown in FIGS. 2 and 3, the determined cartilaginous volumes preferably correspond to at least a portion of the symphysis at the sternomanubrial joint, generally referenced by region 2 in FIG. 2, the synovial joints of the sternabrae, generally referenced by regions L3 and R3 in FIG. 2, and the synchondroses between the first costal cartilage and the manubrium, generally referenced by regions L1 and R1 in FIG. 2, for each respective chest side.

More preferably, the determined cartilaginous volumes correspond to at least a portion of the symphysis at the sternomanubrial joint, the synovial joints of all the sternocostal joints, and the synchondroses between the first costal cartilage and the manubrium, for each respective chest side. These volumes are preferable as they generally correspond to the relevant chondrification processes discussed above in detail.

Thus, for each respective chest side, the total cartilaginous volume is preferably determined through summation of the relevant volumes of the symphysis at the sternomanubrial joint, the synovial joints of the sternabrae, the synchondroses between the first costal cartilage and the manubrium, and other relevant cartilaginous regions. For example, the relevant portions of identified regions L1, 2, and L3 may be summed to determine the cartilaginous volume of the left anterior chest wall and the relevant portions of identified regions R1, 2, and R3 may be summed to determine the cartilaginous volume of the right anterior chest wall.

However, the total cartilaginous volume for each side may be determined through other methods, such as estimation or extrapolation of various cartilaginous volumes within the anterior chest wall or determination of only a single cartilaginous volume within each side, such as only determining the cartilaginous volume of the synovial joints of the sternabrae of each anterior chest wall side.

Furthermore, the total cartilaginous volumes for each side may be determined by generally estimating the volumes, such as by determining that each side has a cartilaginous volume of average, below-average, above-average, or other similar generalized values such that the precise volume of the cartilaginous regions need not be determined. Also, the total cartilaginous volumes for each side may be determined by utilizing the information and/or data generated in steps 104 and 106 relating to various dimensions of the anterior chest wall. For example, these volumes may be determined by calculating the volume of the area provided by the region discussed in step 106 relating to the sternal process, xyphoid process, and C7 and T5 locations, and/or from the dimensions of the sternal prominence discussed in step 104.

In step 104, a cartilaginous volume of the anterior chest wall may also be determined by a displacement of a sternal prominence of the patient. Preferably, step 104 is performed along with steps 100-102 to provide a more detailed assessment of the cartilaginous volumes of the anterior chest wall of the patient. However, step 104 may be performed by itself, such that the variance and assessed breast cancer risk are determined based upon the displacement of the sternal prominence of the patient alone.

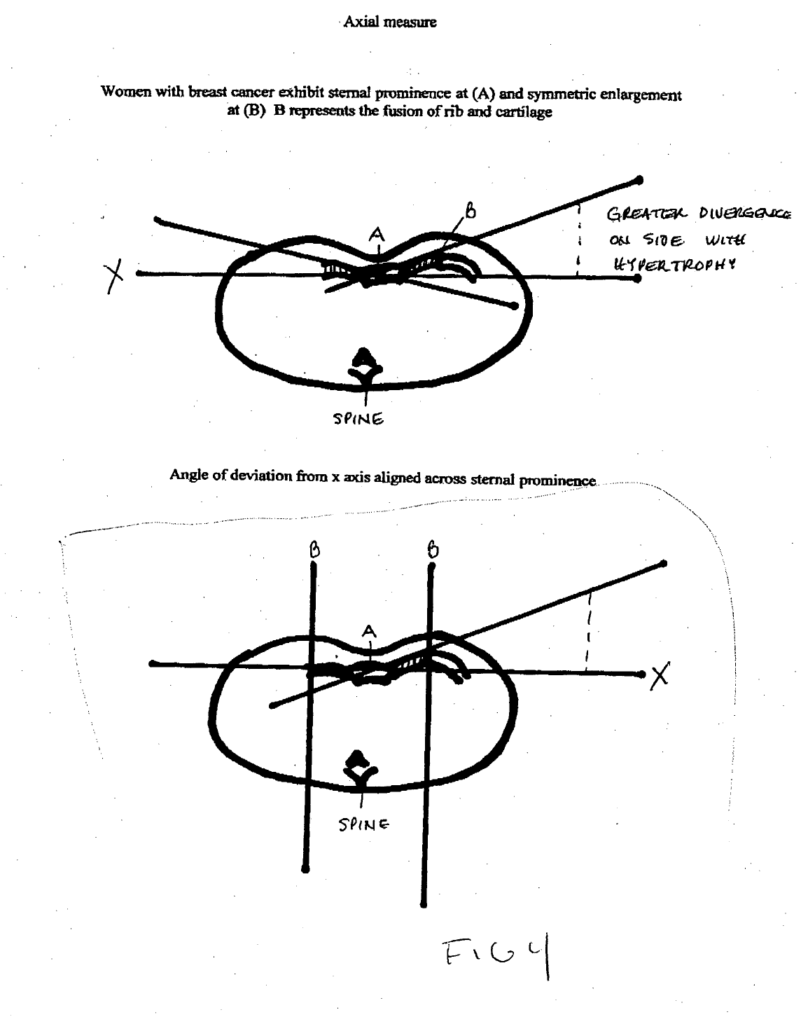

As shown in FIG. 4, the displacement of the sternal prominence is generally determined by the deviation of the sternal prominence from a lateral axis positioned through the patient's manubrium, represented by axis x. Thus, the displacement is generally represented by an angle formed between the lateral axis x at the manubrium and the fusion of rib and cartilage. The displacement of the sternal prominence may be determined utilizing the volumes determined in steps 100 and 102 and/or the displacement of the sternal prominence may be determined without specifically determining the cartilaginous volumes of the left and right anterior chest wall.

Preferably, for each anterior chest wall side, the displacement of the sternal prominence is determined, as the displacement may vary from side to side and thus indicate an asymmetry, as described below. For example, the angles formed between the lateral axis x and the fusion of rib and cartilage that forms the left and right costochondral joints of the patient are each preferably determined for comparison in step 108. However, a single displacement may also be determined for use alone in step 108. Furthermore, the displacement of the sternal prominence need not be specifically quantified, as its determination may be indicated non-numerical indicators such as “average”, “above-average”, etc.

The equipment 10 is preferably utilized to determine the displacement of the sternal prominence. For example, the detecting device 14 may be utilized to determine the position of the sternum and various cartilaginous regions of the anterior chest wall, such as the fusion of rib and cartilage that forms the patient's breast. The computing device 12 may utilize one or more code segments to identify and/or quantify displacement of the sternal prominence from data provided by the detecting device 14. For example, the computing device 14 may form an electronic image of the anterior chest wall and determine a displacement angle for each side of the anterior chest wall by automatically identifying the deviation of the sternal prominence from the lateral axis x. Additionally, medical personnel may manually identify and quantify the displacement of the sternal prominence in a similar manner.

In step 106, a cartilaginous volume of the anterior chest wall may also be determined by an anteroposterior displacement at a sternomanubrial symphysis of the patient. Preferably, step 106 is performed along with steps 100-104 to provide a detailed assessment of the cartilaginous volumes of the anterior chest wall. However, step 106 may be performed by itself, such that the variance and assessed breast cancer risk are determined based upon the anteroposterior displacement of the patient alone, or step 106 may be performed with any combination of the above steps. The anteroposterior displacement may be determined utilizing the volumes determined in steps 100 and 102 and/or the anteroposterior displacement may be determined without specifically determining the cartilaginous volumes of the left and right anterior chest wall.

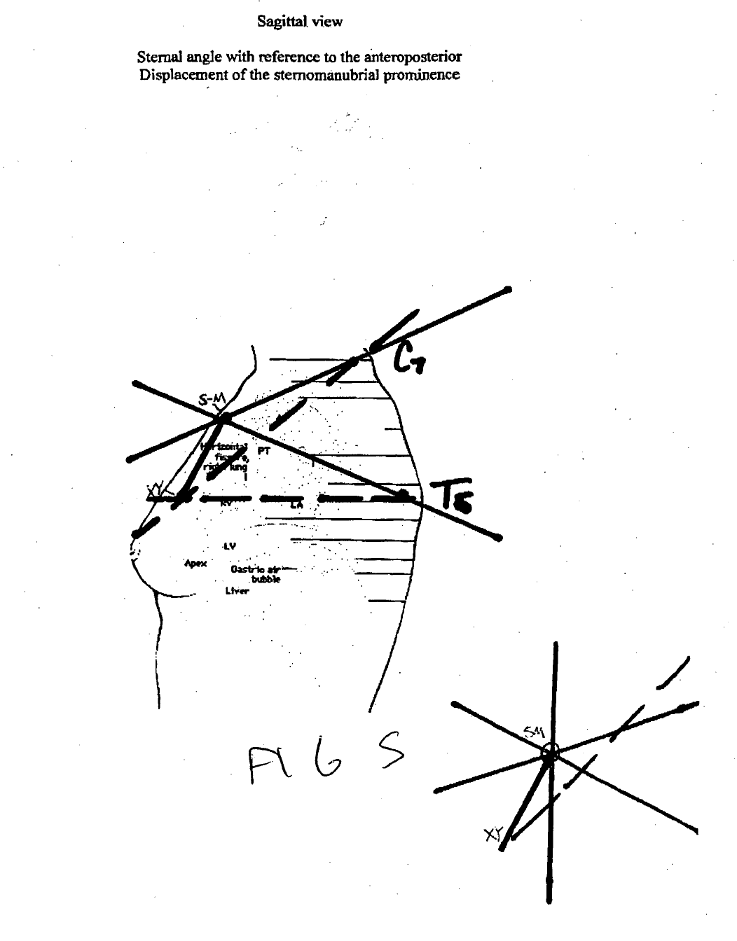

As shown in FIG. 5, the anteroposterior displacement at the sternomanubrial symphysis is generally determined by utilizing a sternal angle formed between the sternal process and spinal references C7 and T5 and a xyphoid angle formed between the xyphoid process and spinal references C7 and T5. A degree of anteroposterior displacement may then be determined by the difference between the sternal angle and xyphoid angle as defined above. However, anteroposterior displacement may be determined through known methods, such as measurements and angles formed between processes and references other than those discussed above.

The equipment 10 is preferably utilized to determine the anteroposterior displacement in a similar manner to step 104. Also, medical personnel may also manually determine the anteroposterior displacement. Furthermore, the anteroposterior displacement need not be specifically quantified, as its determination may be indicated non-numerical indicators such as “average”, “above-average”, etc.

In step 108, the variance is calculated. The variance generally represents asymmetry within the cartilaginous volume of the anterior such wall, such as an asymmetry between the cartilaginous volume of the left anterior chest wall and the cartilaginous volume of the right anterior chest wall. Preferably, the determinations of steps 100-106 are each utilized to calculate the variance. However, one or any combination of the determinations of steps 100-106 may be utilized.

Thus, the variance may be calculated from the difference between the cartilaginous volume of the left side and the cartilaginous volume of the right side of the anterior chest wall. The variance may comprise only a comparison of the left and right anterior chest wall, such as by calculating that the right anterior chest wall has a greater cartilaginous volume than the left anterior chest wall, etc.

Additionally, the variance may include the determination of the displacement of the sternal prominence such that a greater displacement of the sternal prominence results in a greater variance. For example, if the sternal displacement of one side of the patient's anterior chest wall is greater than the other side, the calculated variance is increased. Similarly, if the sternal displacement is otherwise abnormal, the variance may be increased. Thus, if the left and right cartilaginous volumes of the patient's anterior chest wall have generally equal volumes, utilizing the displacement of the sternal prominence provides a variance that represents asymmetry in the desired manner to facilitate assessment of breast cancer risk.

Furthermore, the variance may include the determination of the anteroposterior displacement, such that a greater anteroposterior displacement results in a greater variance. For example, the difference between the sternal angle and the xyphoid angle defined above may be utilized to calculate the variance. In a similar manner to the displacement of the sternal prominence, utilization of the anteroposterior displacement allows the calculation of a variance in situations where a comparison of volumes or sternal prominence displacement alone may indicate that no variance exists. As such, utilization of the determination of the anteroposterior displacement increases accuracy of the calculated variance.

Preferably, the variance is a number that represents the amount of asymmetry within the cartilaginous volume of the anterior chest wall, such as the difference between the cartilaginous volumes of each side of the anterior chest wall. For example, the variance may be a number corresponding to the difference in cubic centimeters between the cartilaginous volumes of the left and right side, or an amalgamation of the difference between the cartilaginous volumes, the sternal prominence displacement, and the anteroposterior displacement.

However, the variance need not be a number or a specific representation of volume. For example, the variance may be an indicator such as “Very High”, “High”, “Average”, etc, a scaled indicator such as a numeral 5 on a variance scale of 1 to 10, or a boolean-type indicator such as “Variance” or “No Variance.”

The variance may be calculated through other methods, such as by determining asymmetry by utilizing the computing device 12 to recognize asymmetry or abnormalities in the cartilaginous volumes of each side. For example, the equipment 10, or an operator of the equipment 10, may identify geometric asymmetry between the determined cartilaginous volumes and determine variance based upon the amount of geometric asymmetry, such as by estimation or utilization of the determined displacements. Thus, asymmetry, and the variance, may exist even though the total determined cartilaginous volume of each side of the relevant portions of the patient's chest are generally equal.

The variance may be also or additionally be calculated based upon stored or received data from the detecting device 14, and/or the variance may be determined by a physician or other medical personnel, by manually identifying asymmetries from the determined volumes or by subtracting the determined volumes from each other.

In step 110, breast cancer risk is assessed utilizing the calculated variance. Preferably, breast cancer risk is assessed based upon the amount of the calculated variance such that a greater variance results in a greater breast cancer risk. For example, the computing device 12 may include a look-up table or similar code element to correlate the variance calculated in step 104 to an assessed breast cancer risk, such as by equating a variance of “Very High” to a risk of “High”, by equating a specific variance volume to a specific risk of breast cancer, and/or by comparing the cartilaginous volumes.

Additionally, a physician or other operator of the equipment 10 may manually assess breast cancer risk based upon the amount of variance. Empirical data and/or statistical data may also be utilized to assess breast cancer risk based upon the calculated variance. For example, data corresponding to average cartilaginous volumes and displacements or cartilaginous volumes and displacements of known breast cancer patients may be utilized to assess breast cancer risk utilizing the calculated variance.

In step 112, data relating to cartilaginous volumes, displacements, calculated variance, assessed risk, and/or any other data relating to the equipment 10, patient, and above steps is stored by the equipment 10. Preferably, the data is stored by the computing device 12 on a computer-readable medium for later access by the equipment 10. For example, the data may be stored on a hard-disk and archived by patient name or identifier such that it may be easily retrieved later for medical or diagnostic use. Furthermore, the computing device 12 may process the stored data to create empirical and/or statistical data for use in step 106.

In step 114, breast cancer risk is assessed utilizing the calculated variance and the stored data from step 112. As breast cancer risk may change over time due to the ability of breast cancer to first appear during generally any portion of a patient's life, it is desirable to repeat steps 100-112 at regular intervals to detect a change in breast cancer risk. As, such, step 114 preferably includes the repetition of steps 100-112 at a time later than originally performed.

Thus, breast cancer risk may be assessed by utilizing the calculated variance of step 108 and a previously calculated variance for the patient. Asymmetry and variance of cartilaginous volumes within the patient's chest may be monitored at intervals over the patient's life span and changes in these volumes over time may be utilized to assess breast cancer risk. For example, the equipment 10, or an operator of the equipment 10, may assess a high risk of breast cancer due to changes in cartilaginous volumes even though any single variance by itself may not provide a risk of breast cancer. Such repeated application of steps 100-112 is desirable as it provides multiple opportunities for early breast cancer risk assessment.

Preferably, the above-steps are performed beginning toward the end of the second decade of the patient's life, when women establish their adult phenotype. As such, the method of the present invention may be utilized to serve as a biomarker for women at risk. Additionally, it will be appreciated that the method and computer program of the present invention may be utilized to clarify surgical decisions. In those women diagnosed with breast cancer, current options include lumpectomy, mastectomy and prophylactic removal of the contralateral breast. Similarly, an indication of risk enables a patient to receive directed efforts towards surveillance and prevention. Furthermore, the present invention may utilize ultrasound or other detecting elements that do not present a biological burden to testing, such as radiation exposure or the removal of tissue.

Although the invention has been described with reference to the preferred embodiment illustrated in the attached drawing figures, it is noted that equivalents may be employed and substitutions made herein without departing from the scope of the invention as recited in the claims.

1. A method for assessing breast cancer risk in a patient, the method comprising:
determining a cartilaginous volume of at least a portion of an anterior chest wall of the patient;
detecting an asymmetry within the cartilaginous volume; and
assessing breast cancer risk utilizing the detected asymmetry.

2. The method of claim 1, wherein the determined cartilaginous volume includes a cartilaginous volume of at least a portion of a left anterior chest wall of the patient and a cartilaginous volume of at least a portion of a right anterior chest wall of the patient.

3. The method of claim 2, wherein the asymmetry is detected by comparing the cartilaginous volumes of the left anterior chest wall and the right anterior chest wall.

4. The method of claim 1, wherein the asymmetry is detected by determining a displacement of a sternal prominence of the patient.

5. The method of claim 1, wherein the asymmetry is detected by determining an anteroposterior displacement of the patient.

6. The method of claim 5, wherein the anteroposterior displacement is determined by ascertaining a sternal angle between spinal locations C7 and T5 of the patient and a xyphoid angle between spinal locations C7 and T5.

7. A method for assessing breast cancer risk in a patient, the method comprising:
determining a cartilaginous volume of at least a portion of a left anterior chest wall of the patient;
determining a cartilaginous volume of at least a portion of a right anterior chest wall of the patient;
comparing the cartilaginous volumes; and
assessing breast cancer risk utilizing the comparison.

8. The method of claim 7, wherein an ultrasound device is utilized to determine the cartilaginous volumes.

9. The method of claim 7, wherein a computing device is utilized to compare the cartilaginous volumes.

10. The method of claim 7, wherein the comparison includes calculating a variance that represents asymmetry between the cartilaginous volume of the left anterior chest wall and the cartilaginous volume of the right anterior chest wall.

11. The method of claim 10, wherein the calculated variance is the difference between the cartilaginous volume of the left anterior chest wall and the cartilaginous volume of the right anterior chest wall.

12. The method of claim 10, wherein breast cancer risk is assessed based upon the amount of the calculated variance such that a greater variance results in a greater breast cancer risk.

13. The method of claim 10, further including comparing the calculated variance to a previously calculated variance.

14. The method of claim 13, further including assessing breast cancer risk utilizing the comparison of the calculated variance to the previously calculated variance.

15. The method of claim 7, wherein the determined cartilaginous volumes include at least a portion of the symphysis at the sternomanubrial joint, the synovial joints of the sternabrae, and the synchondroses between the first costal cartilage and the manubrium.

16. A method for assessing breast cancer risk in a patient, the method comprising:
determining a cartilaginous volume of at least a portion of a left anterior chest wall of the patient;
determining a cartilaginous volume of at least a portion of a right anterior chest wall of the patient;
determining a displacement of a sternal prominence of the patient;
determining an anteroposterior displacement of the patient;
calculating a variance by comparing the cartilaginous volume of the left anterior chest wall and the cartilaginous volume of the right anterior chest wall and ascertaining the magnitude of the determined displacements such that the calculated variance represents asymmetry within the cartilaginous volumes of the anterior chest wall; and
assessing breast cancer risk utilizing the calculated variance such that a greater variance results in a greater breast cancer risk.

17. The method of claim 16, wherein an ultrasound device is utilized to determine the cartilaginous volumes.

18. The method of claim 16, wherein a computing device is utilized to calculate the variance between the cartilaginous volumes and assess breast cancer risk.

19. The method of claim 16, wherein the determined cartilaginous volumes include at least a portion of the symphysis at the sternomanubrial joint, the synovial joints of the sternabrae, and the synchondroses between the first costal cartilage and the manubrium.

20. The method of claim 16, further including comparing the calculated variance to a previously calculated variance and assessing breast cancer risk utilizing the comparison of the calculated variance to the previously calculated variance.

21. A computer program for assessing breast cancer risk in a patient, the computer program stored on a computer-readable medium for operating a computing device and comprising:
a code segment executed by the computing device operable to determine a cartilaginous volume of at least a portion of an anterior chest wall of the patient;
a code segment executed by the computing device operable to detect an asymmetry within the cartilaginous volume; and
a code segment executed by the computing device operable to assess breast cancer risk utilizing the detected asymmetry.

22. The computer program of claim 21, wherein the code segment determines the cartilaginous volume by receiving data from an ultrasound device.

23. The computer program of claim 21, wherein the code segment determines the cartilaginous volume by identifying cartilaginous and non-cartilaginous regions within the anterior chest wall of the patient.

24. The computer program of claim 21, further including a code segment executed by the computing device operable to store data selected from the group consisting of: the cartilaginous volume of the anterior chest wall; the detected asymmetry; the assessed breast cancer risk; and combinations thereof.

25. The computer program of claim 21, wherein the determined cartilaginous volume includes a cartilaginous volume of at least a portion of a left anterior chest wall of the patient and a cartilaginous volume of at least a portion of a right anterior chest wall of the patient, and the code segment detects asymmetry by comparing the cartilaginous volumes of the left anterior chest wall and the right anterior chest wall.

26. The computer program of claim 21, wherein the code segment detects the asymmetry by determining a displacement of a sternal prominence of the patient.

27. The computer program of claim 21, wherein the code segment detects the asymmetry by determining an anteroposterior displacement of the patient.
